(19) United States
(12) Patent Application Publication
(10) Pub. No.: US 2023/0152225 A1
(43) Pub. Date: May 18, 2023

(54) NEW OPTICAL PROPERTY OF A FLUORESCENT TAG

(71) Applicant: BIOCYTEX, Marseille (FR)

(72) Inventor: Philippe PONCELET, Auriol (FR)

(21) Appl. No.: 16/302,213

(22) PCT Filed: May 17, 2017
(86) PCT No.: PCT/EP2017/000593

§ 371 Date: Nov. 16, 2018

(30) Foreign Application Priority Data
May 18, 2016 (FR) 16/00792

(51) Int. Cl. G01N 21/64; G01N 33/533; G01N 33/58

(52) U.S. Cl. CPC G01N 21/6428; G01N 33/533; G01N 33/582

(57) ABSTRACT

Disclosed is a process of detecting at least one molecule of interest in a sample, including the steps of: i) putting the sample in contact with a ligand that is specific to this molecule of interest and that is coupled with a fluorochrome consisting of phycoerythrin or one of its derivatives, ii) excitation of the mixture from step i) by at least one light source with a wavelength between 330 and 425 nm, iii) detecting an emission of light by the mixture following step ii) at a wavelength greater than or equal to 550 nm, and iv) determining whether the molecule of interest is present in the sample in view of the results of step iii).

This patent application claims the priority of the French patent application No. 16/00792 filed on 18 May 2016, which is incorporated herein by reference.

### SCOPE OF INVENTION

This invention concerns the area of fluorescent markers and, more specifically, their use for the detection of biological targets in a sample.

### PRIOR ART

The identification and counting of cellular subpopulations in a biological sample are currently a critical element both in fundamental research as well as in medical testing laboratories for diagnostic purposes.

To this end, various identification methods have been developed, of which, specifically, the flow cytometry (FCM) or FACS (Fluorescence-Activated Cell Sorting) technique is widely used nowadays.

Flow cytofluorometry (F-CFM) is a technique currently employed for single-cell analysis. It makes it possible to separate cellular subpopulations according to their size and to the presence or absence of specific cell surface markers. The detection of these surface markers is therefore accomplished using specific ligands coupled to fluorophores.

In particular, FCM is currently used for the immunological analysis of blood cells in a wealth of diagnostic applications such as the immunophenotyping of leukaemias and lymphomas. These approaches require the use of fluorescent-conjugated monoclonal antibodies (mAbs) and other specific probes conjugated to various fluorochromes in a wide array of colours (wavelengths) within the visible light spectrum.

Thanks to new laser sources available in red (e.g. 633 nm or 645 nm) and more recently in violet (405 nm), of compact size and economically viable, FCM technology has evolved towards instruments currently equipped with 3 lasers, blue (488 nm), red (645 nm) and violet (405 nm), thus boosting the use of fluorochromes. In fact, as these lasers are separated in space and time, it is possible to measure, independently and without interference, the various fluorescent «colours» induced by each laser.

In parallel to this technological evolution, a growing number of new surface markers of cellular or particulate subpopulations of interest have been gradually identified. This has made it necessary to dispose of fluorophores that differ in their optical properties, in order to be able to distinguish the various targeted populations.

\However, the obtaining of new fluorophores useful for differential diagnoses entails complex research work. Indeed, a «good» fluorophore does not equate a simple molecule able to absorb light at a specific wavelength and then to re-emit light, but a molecule that meets precise criteria, such as:
1) A significant extinction coefficient
2) High quantum yield (0.8-0.9)
3) Poor triplet yield
4) Short lifetime of excited state(s)
5) Large Stokes shift
6) Low photobleaching

Considering these requisites, the panel of currently available fluorophores is still limited, especially with respect to certain excitation wavelengths (405 nm laser).

Among the fluorophores used as from the initial FCM applications in cellular phenotyping are phycobiliproteins.

This is a group of accessory pigments that capture light (and that also have immunomodulating, anti-inflammatory, antioxidant and nutritional properties), the properties of which have been broadly elucidated, specifically in patents US 4,542,104 / US 5,055,556 / US 4,859,582 and EP 0076695 B1.

Even today, these fluorophores are often used for FCM analyses and other cell identification methods.

Indeed, these molecules are able to generate fluorescence spectral signals complementary to that of fluorescein, the main fluorochrome known to be excitable by the 488 nm blue laser. However, this type of laser has long been the only source of light excitation equipped with the first cytofluorometers.

Among phycobiliproteins, phycoerythrin (PE) in particular, has rapidly sparked greater interest due to its high quantum yield (much higher than that of fluorescein isothiocyanate (FITC)), a very useful secondary excitation peak at around 488 nm (in addition to a higher peak at around 560 nm) and excellent complementarity of its emission spectrum as compared to that of the FITC. These characteristics have, to this day, made this a key fluorochrome in FCM-based immunophenotyping.

Moreover, the growing demand for new fluorescent « colours » compatible with the 488 nm laser has encouraged the development PE-tandems (e.g. PE-Texas red or ECD, PE-Cy5, PE-Cy7 ...) enabling 5-colours immunophenotyping with a single laser. Lastly, to this greater application in FCM is added the most recent one of FCM-based multiplex immunoassays in which PE is used more often as an indicator of every single fluoroimmunoassay in the form of mAb conjugates (« reporter conjugates»).

For years, PE has therefore been a particularly interesting fluorochrome not only for FCM, but more broadly for all analytical, detection or separation methods based on receptor/ligand molecular interactions.

As illustrated in FIG. 1, the excitation (absorption) spectrum of PE is generally described as from 450 nm and includes, first of all, a secondary peak at 490 nm followed by a greater peak at 560 nm. Hence, PE has always been used with excitation light sources with a wavelength between 488 and 561 nm, and there was nothing to suggest that it would constitute a satisfying fluorochrome with an excitation wavelength at around 405 nm.

However, surprisingly, the inventors of the present invention have now emphasised that it is possible to use phycoerythrin as a fluorochrome, not only with lasers emitting between 488 and 561 nm, but also with a violet laser (405 nm).

The present invention therefore concerns a new use of PE and its derivatives, such as its tandem derivatives, by using an excitation wavelength close to 405 nm instead of the conventional wavelength close to 488 nm or 560 nm.

### SUMMARY OF THE INVENTION

According to a first object, the invention concerns a method for detecting at least one molecule of interest in a sample, comprising the steps of :
i) Contacting the sample with a ligand that specifically binds the molecule of interest, said ligand being coupled to a fluorochrome consisting of phycoerythrin or a derivative thereof,
ii) Exciting the mixture of step i) by at least one light source with a wavelength range between 330 nm and 425 nm,
iii) Detecting light emitted by the mixture excited in step ii) at a wavelength longer than or equal to 550 nm, and
iv) Determining the presence or absence of the molecule of interest in the sample based on the results obtained in step iii).

The method of the invention can therefore be implemented on an appropriate system comprising at least one ligand conjugated to a fluorochrome consisting of phycoerythrin or one of its derivatives, at least one light source emitting a light with a wavelength between 330 nm and 425 nm and at least one detector of the light emitted by the excited fluorochrome.

Furthermore, the method may also aim at the simultaneous detection of a second, third, fourth and fifth (or umpteenth) molecule of interest in a same sample.

Another object of the invention concerns the use of a fluorochrome consisting of phycoerythrin or one of its derivatives for the detection of at least one molecule of interest in a sample, characterised in that the excitation wavelength used is between 330 nm and 425 nm, preferably at 405 nm.

### DESCRIPTION OF THE DRAWINGS

FIG. 1 shows the absorption/excitation spectra (dotted dashed lines) and the emission spectra (solid lines and shaded under the curve areas) of phycoerythrin (R-PE) as well as three of its tandems, namely R-PE-TexasRed, R-PE-Cy5.5 and R-PE-Cy7 (adapted from Fluorescence SpectraViewer, Thermofisher Scientific). The wavelengths (in nm) are presented on the x-axis and the y-axis illustrates the relative absorbance or emission intensities (in %). The curves are outlined only as from 400-450 nm with obvious excitation peaks at around 490, 530 and 560 nm for R-PE. In addition to these three excitation peaks, additional excitation peaks are visible for R-PE-Cy5.5 (660 nm) and R-PE-Cy7 (760 nm). The emission spectrum of R-PE is a Gaussian function centred at 575 nm. The emission spectra of tandems are shifted to the longest wavelengths and are centred at 615 nm (R-PE-Texas-Red), 694 nm (R-PE-Cy5.5) and 776 nm (R-PE-Cy7).

FIGS. 2 to 8 illustrate the absence of leakage of phycoerythrin excited by the blue laser in the read channel of the violet laser. Polystyrene beads coated with in increasing amounts of R-PE serve as a calibrator and are excited by the blue and violet lasers. FL2 corresponds to the signals from the blue laser (488 nm) passing through a 575 BP 30 filter, behind a 595 DC SP dichroic mirror. FL10 corresponds to the signals from the violet laser (405 nm) passing through an identical 575 BP 30 filter, after deflection by a 480 DC SP dichroic mirror.
The nominal powers of the blue and violet lasers are respectively equal to 22 mW and 40 mW (FIGS. 2 and 5). The bead singlets are initially preselected by the «beads» region in a double scatter analysis and their FL2 and FL10 fluorescences are analysed in a correlated way.
The nominal power of the blue laser is reduced to 10 mW (FIG. 3) and then 5mW (FIG. 4), whereas the nominal power of the violet laser is maintained at 40 mW. The nominal power of the violet laser is reduced to 10 mW (FIG. 6) and then 5 mW (FIG. 7), whereas the nominal power of the blue laser is maintained at 22 mW. The violet laser was blocked by an iris during analysis that makes it possible for it to artificially drop its nominal power from 40 mW to 0 mW; the nominal power of the blue laser is kept constant at 22 mW (FIG. 8).

FIG. 9 illustrates the ability of PE-Cy5.5 excited by a violet laser to distinguish between a strongly stained and a lightly stained sample. Some capture beads, coated or not with a variable amount of goat anti-mouse antibodies are exposed to the anti-CD45 antibody conjugated to PE-Cy5.5 The sample is successively excited by the violet and blue lasers, whereby separations in space and time enable differentiated analyses of the fluorescence signals emitted from each laser. FL4 corresponds to the signals from the blue laser (488 nm) passing through a 695 BP 30 filter, behind a 730 DC SP dichroic mirror to collect the fluorescence of PE-Cy5.5. FL10 corresponds to the signals from the violet laser (405 nm) passing through an identical filter (695 BP 30), after deflection by a 480 DC SP dichroic mirror.

FIG. 10 shows the identification of blood cell subpopulations via flow cytometry of normal haematopoietic cells using the CD45 marker. The excitation of the anti-CD45-PE-conjugated antibody at 488 nm is outlined in figures (A), (C) and (E). The excitation of the anti-CD45-PE-conjugated antibody at 405 nm is outlined in figures (B), (D) and (F). The tracking and quantification of blood cell subpopulations are represented by figures (A), (B), (C) and (D). Figures (E) and (F) are a representation in combined single-parameter histograms of the 4 subpopulations: lymphocytes (Ly), monocytes (Mo), granulocytes (PMNs) and residual red blood cells (Ery).

FIG. 11 shows the identification of blood cell subpopulations via flow cytometry of normal haematopoietic cells and leukaemias using the CD45 marker. LP1 cells, serving as a model of abnormal cells having low level of CD45 expression, have been added at a rate of 10% compared to the normal white blood cells. The excitation of the anti-CD45-PE-conjugated antibody at 488 nm is outlined in figures (A), (C) and (E). The excitation of the anti-CD45-PE-conjugated antibody at 405 nm is outlined in figures (B), (D) and (F). The tracking and quantification of blood cell subpopulations supplemented with LP1 cells are represented by figures (A), (B), (C) and (D). Figures (C) and (D) display only the leukaemic blasts. Figures (E) and (F) are a representation in combined single-parameter histograms of the 5 subpopulations: lymphocytes (Ly), monocytes (Mo), granulocytes (PMNs), residual red blood cells (Ery) and the leukaemia cells of the LP1 cell line (LP1).

FIG. 12 shows the identification of blood cell subpopulations via flow cytometry of normal and leukaemic haematopoietic cells using the CD45 and CD33 markers. HL60 and NB4 cells, serving as a model of abnormal Acute Myeloid Leukaemia (AML) having low level of CD45 expression (CD45 low), have been added in a quantity of 50,000 cells/sample. The excitation of the anti-CD33-PE-Cy7 conjugated antibody at 488 nm is outlined in figure (A). The excitation of the anti-CD33-PE-Cy7 conjugated antibody at 405 nm is outlined in figure (B). The tracking and the statistics associated with blood cell subpopulations supplemented with HL60 and NB4 cells are represented by figures (A) and (B). Figures (C) and (D) are a two-colour bi-parametric representation of the distribution of normal blood cell subpopulations including neutrophils (Neu and PMN2), lymphocytes (Ly), residual red blood cells (RBC) and added leukaemic cells (HL60 and NB4).

### DETAILED DESCRIPTION OF THE INVENTION

A first object of the invention concerns a method for detecting at least one molecule of interest in a sample, comprising the steps of:
i) Contacting the sample with a ligand that specifically binds the molecule of interest, said ligand being conjugated to a fluorochrome consisting of phycoerythrin or a derivative thereof,
ii) Exciting the mixture of step i) by at least one light source with a wavelength range between 330 and 425 nm,
iii) Detecting light emitted by the mixture excited in step ii) at a wavelength longer than or equal to 550 nm, and
iv) Determining the presence or absence of the molecule of interest in the sample based on the results of step iii).

The inventors have underlined, as shown in the examples below, that phycoerythrin has fluorescence properties following excitation by a laser at 405 nm, such as to enable its use as a fluorochrome in connection with such a laser.

The phrase «molecule of interest» refers to a biological marker, the presence of which is suspected in a sample. Such a biological marker can be of proteic, nucleic, glucidic, lipidic or glycolipidic nature.

Preferably, the biological marker detected by the method of the invention is a protein, a lipid, a carbohydrate, a lipid or glycolipid structure or a carbohydrate unit.

Preferably, the biological marker detected by the method of the invention is a protein.

The term «sample» refers to any type of sample, chiefly biological samples consisting of tissues (muscle, bone, organ, etc...), body fluids (blood, saliva, urine, tears, sperm, milk, bronchoalveolar fluid, pleural fluid, cerebrospinal fluid, etc...), liquid suspensions containing molecules of interest, specifically particles, in particular cells, eucaryotes or procaryotes, such as in vitro culture media or fermentation fluids, bathing waters or drinking water, injectable liquids.

Preferably, the sample used in the detection method of the invention includes or is likely to include cells, procaryotes or eucaryotes, preferably eucaryotes, preferably derived from animal body, in particular from human body. In such a case, the molecule of interest to be detected can be intracellular or anchored to the cell plasma membrane.

Preferably, the molecule of interest is present in the sample at a density of at least 1,000 copies per cell, more preferably of at least 2,500 copies per cell, still more preferably of at least 5,000 copies per cell.

Preferably, the molecule of interest is present on/in the cells of the sample at a density ranging from 1,000 to more than 2,000,000 copies per cell, more preferably from 7,500 to 1,500,000 copies per cell, more preferably still from 10,000 to 150,000 copies per cell.

According to a particular embodiment of the invention, the molecule of interest is present on/in the cells of the sample at a density of at least 7,500 copies per cell.

The term «ligand» refers to a macromolecule able to physically and reversibly bind to the targeted molecule of interest. This interaction is possible thanks to the existence of zones of complementarity between the molecule of interest and its specific ligand. The binding between ligand and the molecule of interest is done by establishing non-covalent bonds, for instance via electrostatic bonds, Van Der Waals forces, ionic or hydrogen bonds and also thanks to hydrophobic interactions. When the molecule of interest is a protein, the ligand may in this case be an antibody, an antibody fragment or derivative structures (Fab, Fab′2, nanobody, aptamer, affimer...). When the molecule of interest is a glycosylated determinant, the ligand can be a lectin or a bacterial toxin (e.g. FLAER for its affinity to glycosylphosphatidylinositol anchors of numerous membrane antigens). When the molecule of interest is a lipid (e.g. phosphatidylserine, ceramide, GD2, GD3 ...), the ligand can be an affine protein such as (but not limited to) an annexin, lactadherin, cholera toxin ... Lastly, when the molecule of interest is a hapten (biotin, DNP, poly-His ...), the avidin, the streptavidin of the anti-hapten antibodies can be involved. Lastly, for any ligand, one of its receptors may be used as a probe. Irrespective of the nature of the ligand or of the molecule of interest, the stability of association between the ligand and the molecule of interest is quantifiable by determining the association or equilibrium constant (Ka) specific of this bond. This constant corresponds to the ratio between the concentration of the ligand-molecule of interest complex and the product of the free ligand concentration and the free molecule of interest. For this bond to be qualified as specific, it must be greater than or equal to 10⁶ L.mol⁻¹.

Preferably, the ligand contacted with the sample in the invention detection method is an antibody.

The term «fluorochrome» refers to any type of compound able, following irradiation by a light beam in a range of wavelengths from ultraviolet to infrared, to emit a photon of lower-energy in return. Irradiation generates fluorescence that is typically an illumination. The emission of photons takes place during the electronic transition of a molecule from an excited state resulting from the absorption of light energy to a ground state (relaxation). Thus, the absorption of a photon by a fluorochrome leads to the emission of another photon of longer wavelength and lower energy as compared to those of the exciter photon. Depending on the wavelength and energy of the light beam, the fluorochrome may reach various excited states.

Each fluorochrome has its own specific absorption spectrum, excitation spectrum and emission spectrum. The absorption spectrum of a fluorochrome is defined by its range of absorbed wavelengths, regardless of its excitation. The excitation spectrum of a fluorochrome is defined by its range of wavelengths effectively inducing excitation, also reflecting the range of excited states that the fluorochrome can achieve. When the fluorochrome is illuminated (excited) at a wavelength corresponding to its maximum excitation, the emission of the fluorochrome is then maximum. If, conversely, the fluorochrome is excited at a wavelength other than its maximum excitation, the emission length is therefore the same, however, the fluorescence intensity is lower.

The fluorescent light efficiency for a given fluorochrome is determined by quantum yield phi (ϕ) defined by the ratio between the number of photons emitted and the number of photons absorbed by the fluorochrome. Hence, this quantum yield allows to differentiate the absorption and the excitation at a given wavelength. Typically, fluorochromes have quantum yields between 0.1 and 1. As the quantum yield Φ is independent of the excitation wavelength (Kasha’s rule), the emission spectral shape of a fluorochrome is invariant, regardless of the wavelength of the excitation light source. Nevertheless, the intensity of the emission spectrum of a fluorochrome varies according to the excitation wavelength.

The absorption intensity, or extinction coefficient ε, or specific absorbance, reflects the absorption probability; the higher the coefficient ε is, the higher the fluorescence at the same light intensity.

The fluorescence intensity or brightness of a fluorochrome is determined by the product of the extinction coefficient and the quantum yield. The higher this product is, the brighter the fluorochrome is.

The terms fluorophore, fluorochrome or fluorescent probe are equivalent and may be used interchangeably.

The term «phycoerythrin», refers to proteins belonging to the family of phycobiliproteins extracted from cyanobacteria, red algae and certain Cryptophytes. Phycobiliproteins are composed of an apoprotein covalently bound to a chromophore known as phycobilin. An apoprotein has a monomeric structure comprising two distinct polypeptides referred to as subunits α and β, organised in the form of a trimer (αβ)₃ or a hexamer (αβ)₆. Such typical complexes may also contain a third type of subunit, the γ-chain.

The absorbance characteristics of phycobiliproteins are due to the existence of open-chain tetrapyrrole prosthetic groups that are bound to the subunits α, β and γ through a thioether bond with a cysteine residue of at least one of the polypeptides.

Phycoerythrobilin (PEB) and phycourobilin (PUB) constitute the two principal types of tetrapyrrole prosthetic groups of phycoerythrins extracted from the red algae.

The number of PEB and PUB groups may vary according to the cellular origin of phycoerythrin. Thus, five classes of phycoerythrin have been identified: C-phycoerythrin (C-PE) binds two PEBs per subunit α and three PEBs per subunit β, CU-phycoerythrin (CU-PE) binds two PEBs per subunit α and three PEBs and one PUB per subunit β, b-phycoerythrin (b-PE) binds two PEBs per subunit α and four PEBs per subunit β, B-phycoerythrin (B-PE) binds two PEBs per subunit α, three PEBs per subunit β and two PEBs and two PUBs per subunit γ, and R-phycoerythrin (R-PE) binds two PEBs per subunit α, two PEBs and one PUB per subunit β and one PEB and three PUBs per subunit γ.

The ratio between the number of PEB and PUB groups defines the spectroscopic properties of the various phycoerythrins. Therefore, the maximum excitation peak of PEs generally varies from about 545 nm (b-PE) to about 565 nm (R-PE).

According to a particular embodiment, the fluorochrome is phycoerythrin-R (R-PE), and preferably the PE used in the detection method according to the invention is extracted from red algae.

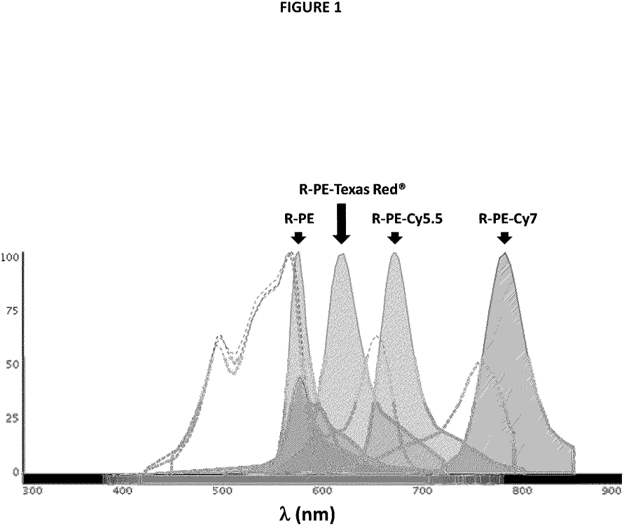

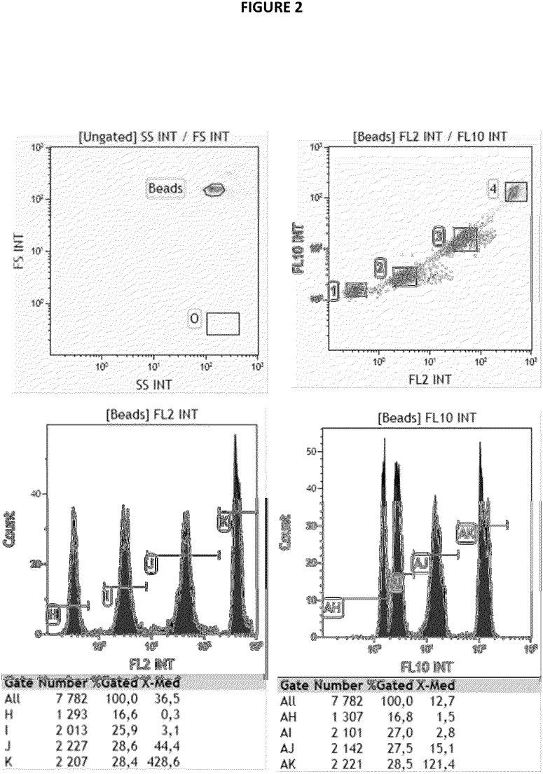

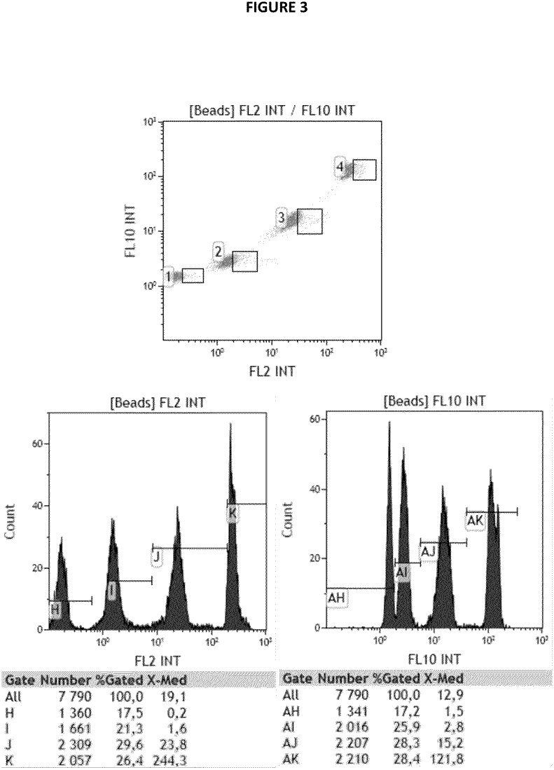

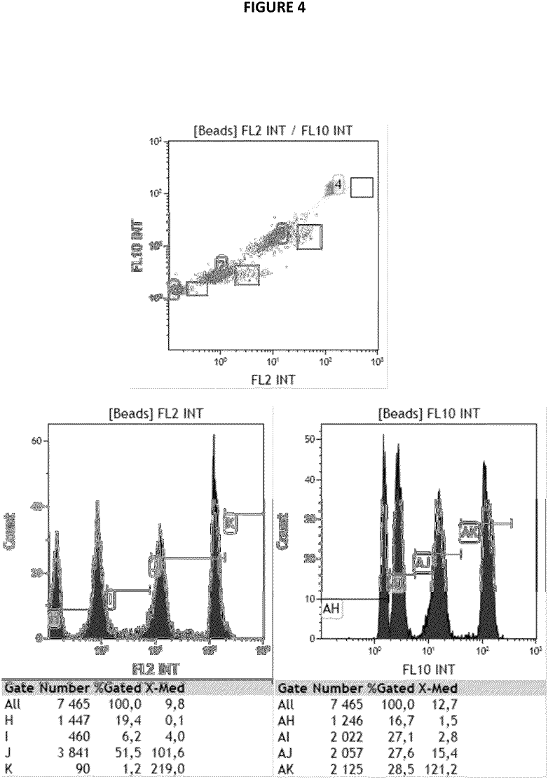

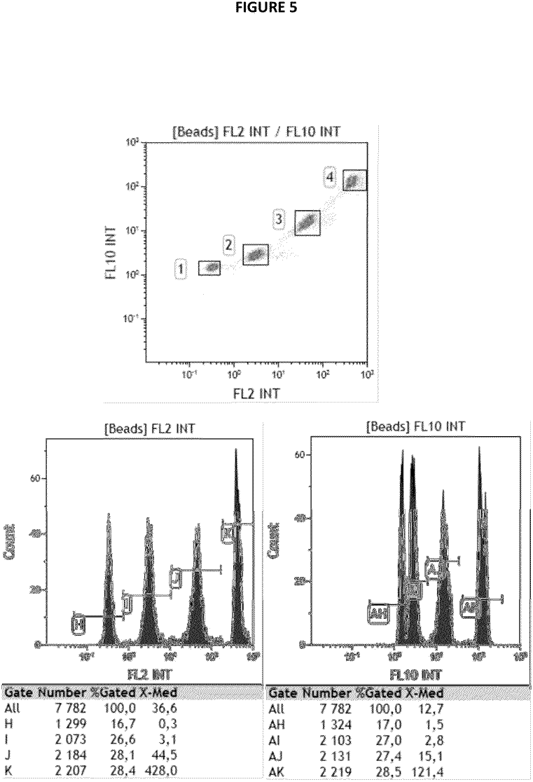

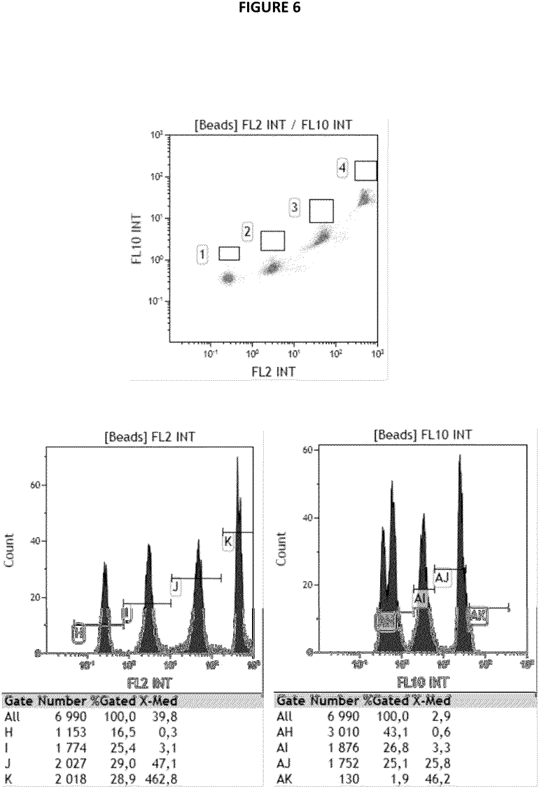

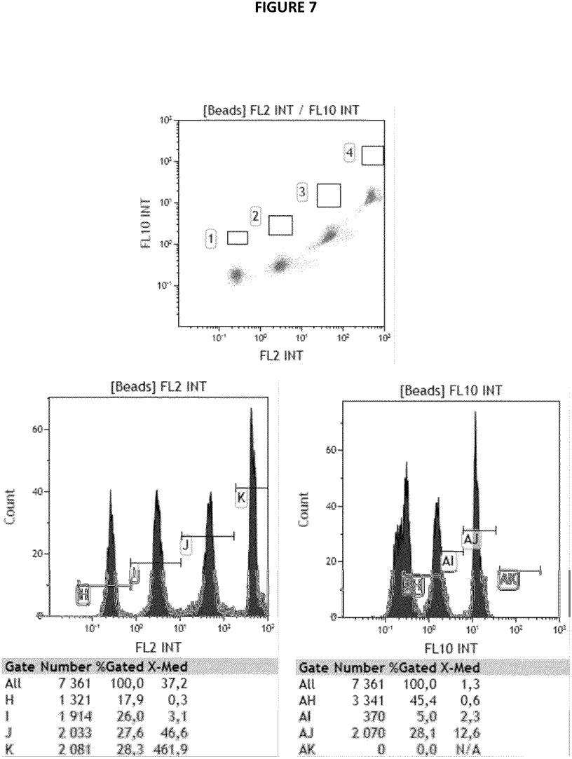

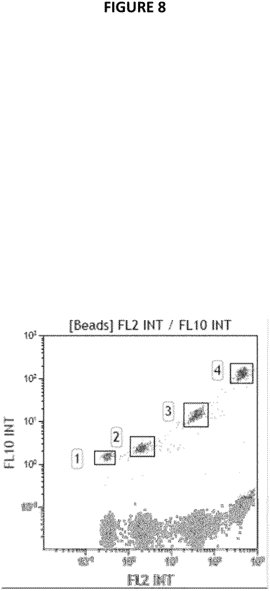

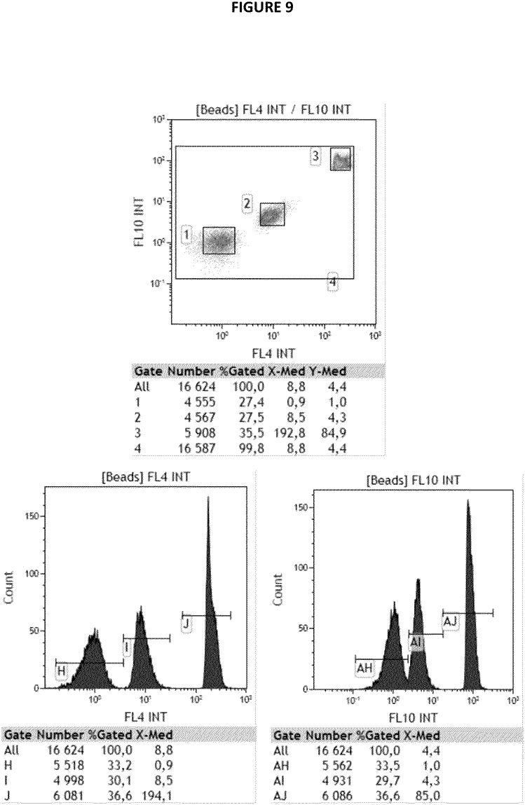

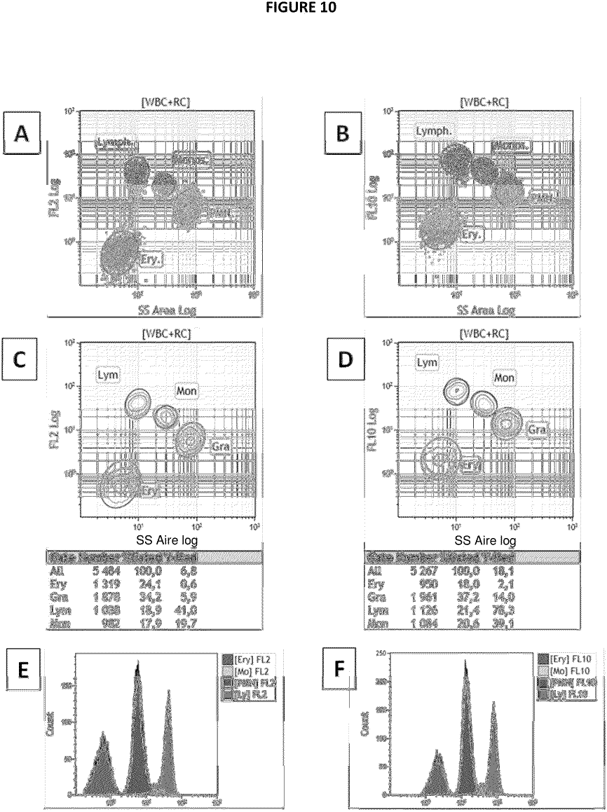

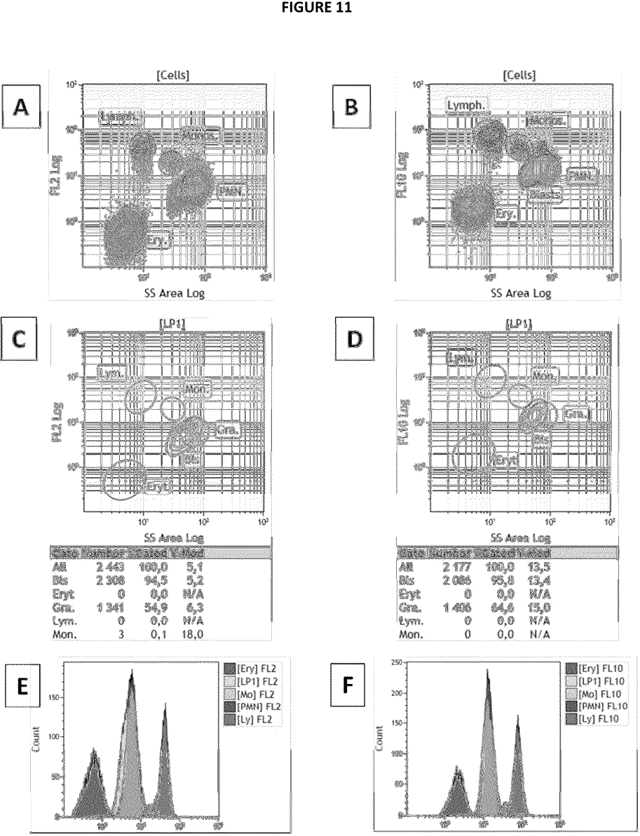

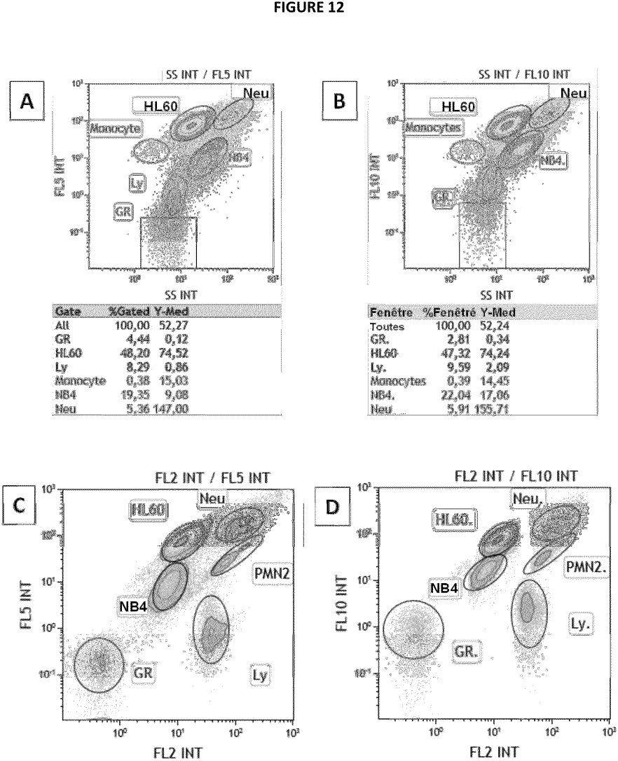

As illustrated in FIG. 1, the absorption spectrum of phycoerythrin is measured over a range between 400-450 nm and 600 nm and displays 3 major absorption peaks corresponding to maximum absorption intensities at around 490 nm, 530 nm and 560 nm. FCM literature and all the textbooks instruct the user to select one of these 3 absorption peaks, taking advantage of the most accessible laser sources used in FCM that are the blue laser at 488 nm, present in the quasi entirety of devices currently available on the market, the green laser at 530 nm and, more recently a yellow laser at 560 nm. However, it was as yet unknown that the very low absorption at about 400 nm would allow satisfying satisfactory excitation of PE and therefore the achievement of a fluorescence applicable to the identification of haematological cell subpopulations of potential diagnostic interest, such as leukaemic blasts in human blood, and that can also be repeated. Indeed, a fluorochrome absorbing light at a given wavelength is not necessarily efficiently excited at this wavelength. The excitation spectrum of a substance is achieved using a spectrofluorometer by measuring the fluorescence emitted at a fixed wavelength and allowing the excitation wavelength to vary.

The fluorescence emission spectrum always shifts to longer wavelengths in relative to the absorption spectrum. The emission spectrum of a substance is achieved by measuring the fluorescence emitted at different emission wavelengths, the substance being excited with a fixed wavelength. Unlike most fluorochromes, the emission spectrum of PE does not correspond to a simple shift towards longer wavelengths of its absorption spectrum. As detailed in FIG. 1, the emission spectrum of PE is a Gaussian function centred on 575 nm, whereas the excitation spectrum shows a complex shape with multiple maxima. However, the emission of PE can sometimes be detected as from 550 nm. Whereas the shape of the fluorescence emission spectrum does not depend on the excitation wavelength, the intensity of fluorescence radiation is directly proportional to the excitation beam power absorbed by the fluorophore.

Based on this spectrum, the skilled artisan has always used the wavelengths corresponding to optimal or slightly sub-optimal excitation intensities of this fluorophore, with either a blue light source, with a wavelength between 480 nm and 500 nm, or with a green/yellow light source, with a wavelength between 530 and 565 nm.

The chromophores of PEs extracted from Cryptophytes also contain cryptoviolin (CV). This is particularly the case of phycoerythrin-544 (PE-544) that binds two PEBs and two CVs per subunit α and one PEB per subunit. β. Phycoerythrin-555 (PE-555) and phycoerythrin-568 (PE-568) bind the PEB to the two subunit types α and β. It appears that PEs extracted from Cryptophytes do not have any subunit γ. The excitation spectrum of PEs extracted from Cryptophytes generally varies between 544 nm (PE-544) and 568 nm (PE-568).

According to a specific embodiment, the fluorochrome is phycoerythrin-R (R-PE), and preferably the PE used in the detection method according to the invention is extracted from red algae.

Commercial forms of R-PE are available as lyophilisates in an already activated form (e.g. Lyo (SMCC)-RPE, ref. L1 or L1SM distributed by FEBICO) or in liquid form (e.g. PhycoPro™RPE, ref. PB32 distributed by PROZYME or R-phycoerythrin, ref. P801 distributed by INVITROGEN) or more often still in the form of ammonium sulfate precipitate (FEBICO, PROZYME) to be re-solubilised and activated immediately before use.

The coupling of the fluorochrome with the ligand is executed in accordance with well-known techniques to the skilled artisan (M. Roederer, Conjugation of monoclonal antibodies (August, 2004; http://www.drmr.com/abcon/)). Briefly, after extraction and purification of PE, the latter is chemically activated enabling it to be reactive towards thiol groups. The ligand undergoes a reduction phase that enables the release of thiol groups ready to react. Hence, the covalent coupling of the ligand with PE can be carried out.

Commercial kits are available that contain all reagents required to execute the conjugation of PE with the ligand (e.g. PhycoLink® R-Phycoerythrin Conjugation Kit, ref. PJ31K marketed by PROZYME, R-Phycoerythrin Conjugation Kit, ref. ab102918 marketed by AbCAM).

The phrase «phycoerythrin derivatives» refers to the tandem complexes formed between PE and another fluorescent compound. The establishment of a covalent bond between PE and this second fluorescent compound allows energy transfer by resonance from the PE to this second fluorescent compound. In this case, PE is referred to as donor fluorochrome and the second fluorescent compound is referred to as acceptor fluorochrome. The purpose of these tandems is to obtain a fluorescence resonance energy transfer (FRET) and accordingly, a large-scale shift between the primary excitation of the donor fluorochrome and the emission of the acceptor fluorochrome (Stokes shift). Thus, PE tandem complexes have an excitation/absorption spectrum corresponding to that of PE and an emission spectrum corresponding to that of the second fluorescent compound. Such tandem complexes currently available on the market are selected from PE-TEXAS-RED™, PE-Cy5™, PE-Cy5.5™, PE-Cy7™, PE-Alexafluor™610, PE-Alexafluor™647, PE-DyLight™594, PE-Dyomics™590 and RT665™ as well as all PE-X tandems wherein X=any fluorescent compound able, after excitation by absorption of the photon emitted by PE, to emit a photon with a wavelength higher than that emitted by PE alone (i.e. higher than 575 nm).

The Y-PE tandem complexes wherein Y=any donor fluorochrome emitting, following excitation, a photon captured by PE (i.e. acceptor fluorochrome) are excluded from the definition of phycoerythrin derivatives for the purposes of the present invention. In other words, in the PE-X tandems according to the invention, PE is always the donor fluorochrome.

Preferably, the step of contacting the sample with the specific ligand of the molecule of interest is executed in a liquid medium, preferably in aqueous solution.

The term «light source» refers to a device able to emit a beam of monochromatic light. The light source can be a laser, an arc lamp or a light-emitting diode (LED). Arc lamps (e.g.: mercury, xenon-mercury) are light sources that supply an incoherent light of a few milliwatts. They emit at multiple wavelengths that must be filtered in order to select the required wavelengths. For this reason, most flow cytometers have one or more lasers as light source/s. A laser supplies a coherent light from a few milliwatts to several watts, with a narrow, well-defined and specific wavelength. A plethora of different laser types is currently available. Some lasers currently and historically used in FCM include argon lasers (351, 454, 488, 514 nm), krypton lasers (406, 488, 532, 630 nm), helium-neon lasers (632 nm), helium cadmium lasers (325, 441 nm), Yag lasers (532 nm) and violet lasers (405 nm). Recent technological progress has brought about the availability of lasers that are much less unwieldy and endowed with an extended lifespan thanks to what is referred to as «solid-state» or DPSS (diode-pumped solid state) technology, to be found today in a wide array of wavelengths similar to conventional gas lasers, in particular the DPSS laser at 488 nm, 635 nm and 405 nm. Nowadays, the flow cytofluorometers available on the market are currently equipped with 3 blue, violet and red lasers and this evolution has resulted in the development of new fluorochromes ideally excited by red and violet lasers, in addition to the historical list of fluorochromes excitable by the blue laser, including PE and its tandems. Lastly, the rapid and recent evolution of CD-ROM drives and DVD players has boosted laser diode systems endowed with characteristics and power that make them usable as excitation sources as an alternative to DPSS lasers, with a much lower cost (1:100 or even 1:1,000 ratio) owing to their industrial mass production series. Thus, on the market it is possible to find red laser diodes (635 nm, included in CD-ROM drives), blue laser diodes (450 nm, included in DVD players) and now blue-violet laser diodes (405 nm, included in blue-ray players). It is relevant to observe that on the market there are no performing and cheap diode lasers centred at 488 nm, and as a result, a device that only comprises one laser source (or laser diode) at 370 nm or 405 nm, or two violet and red sources (405 and 635 nm) shall be both more compact (compact design of the laser diodes) and cheaper than a device claiming the presence of a laser source at 488 nm or 530 nm or 560 nm. The use of a blue-violet diode laser (line at 405 nm, http://www.lasercomponents.com/fileadmin/user_upload/home/Datasheets/kimmon/blue-violet-laser-specs.pdf) is also contemplated for diverse biomedical methodologies, such as, for example, FCM. Specific economic and industrial considerations (reliability, greater compactness, ease of integration) make the red and violet laser diodes (635 and 405 nm) particularly attractive for the development of biomedical analytical instruments. This has become all the more interesting with the development of new «point of care» diagnostic tests to be performed in close vicinity to the patient, in medical centres, in pharmacies or other healthcare facilities, provided the result is readily available. Such tests aim to foster rapid patient care and the immediate prescription of appropriate treatment.

Preferably, said at least one light source used for the excitation of the mixture outlined in step i) in the method of the invention has a wavelength between 330 and 425 nm, preferably between 350 and 420 nm, and preferably still between 360 and 410 nm.

According to a particular embodiment, said at least one light source used for the excitation of the mixture of step i) in the method of the invention has a wavelength between 400 and 410 nm, and more preferably 405 nm.

According to another embodiment, said at least one light source used for the excitation of the mixture of step i) in the method of the invention is a violet laser or a violet laser diode emitting at a wavelength of 405 nm.

Step ii) relating to the excitation of the mixture of step i) corresponds to an illumination of the latter by at least one light source.

When the sample contains the molecule of interest and the ligand conjugated to a fluorochrome has bound itself on the latter, the light emitted by the mixture following step ii) is collected at a wavelength equal to or longer than 550 nm, that corresponds to the start of the emission spectrum of PE alone. For instance, the maximum emission wavelength of PE-TEXASRED™, PE-Cy5™, PE-Cy5.5™, PE-Cy7™, PE-Alexafluor™610, PE-Alexafluor™647, PE-DyLight™594, PE-Dyomics™590 and RT665™ tandems are respectively equal to 613 nm, 670 nm, 690 nm, 775 nm, 628 nm, 665 nm, 618 nm, 599 nm and 680 nm. These emission wavelengths can vary by a few nanometers depending on the source and on the protocol of fluorophore extraction/purification. They represent the centre of the optimal fluorescence collection area, the width of which is frequently between 20 and 50 nm, in most cases 30 nm, based on the presence or absence in the analysis of other fluorochromes of close spectra.

A considerable advantage stemming from the excitation of PE and its derivatives with a light source of a wavelength between 360 and 410 nm, preferably at 405 nm, is that this generates a very significant Stokes shift between the excitation wavelength and the emission wavelength, thus making it possible to use fluorescence filters (for PE) that are much less efficient and expensive than those claimed by an excitation with a light source of a wavelength at 488 nm or even more at 530 nm or 560 nm.

Step iii) of detecting the light emitted by the mixture following excitation in step ii) is performed using an optical detector that can be a photomultiplier tube (PMT). In this case, the signal emitted in the form of photons is converted by the photomultiplier tube into a quantifiable electrical signal.

The method of the invention further comprises the simultaneous identification of at least a second, but also potentially at least a third, a fourth, and even a fifth (or umpteenth) molecule of interest in the sample.

Such an identification relies particularly on the use of a first ligand coupled to PE and of a second, third, fourth, or even fifth ligand conjugated to one of its tandems. The light source of a wavelength between 330 and 425 nm, preferably at 405 nm, makes it possible to simultaneously excite PE and its tandem(s), which provide fluorescence of shifted spectra.

According to a particular embodiment, the simultaneous identification of a second molecule of interest can be carried out with the aid of a second fluorophore excitable by a light source of the same wavelength as that used to excite PE or one of its derivatives, but with an emission spectrum different from that of PE or one of its derivatives.

Hence, the light source of a wavelength between 330 and 425 nm, preferably at 405 nm, makes it possible to simultaneously excite PE and/or one of its tandems and fluorochromes specifically designed to be excitable at such wavelengths. These fluorochromes comprise, in particular, SYTO40, DAPI, DyLight® 405, Brilliant Violet 421™, HiLyte Fluor™ 405, Pacific Blue™, Pacific Orange™, Cascade® Blue, Alexa Fluor® 405, eFluor® 450, BD™Horizon™ V450, VioBlue®, VioGreen™, Krome Orange™, Calcein Violet 450 AM™, Zombie Violet™, Aminomethylcoumarin (AMCA) or even certain Q-dot® 565/605/625/655/705/800.

This strategy also makes it possible to combine royalty-free fluorochromes, such as DAPI, and PE or its tandems, which are therefore cheaper and more accessible for industrial applications than the fluorochromes recently developed for working with a violet laser at 405 nm.

According to yet another particular embodiment, the identification of a second molecule of interest can be carried out using a second fluorophore that can be excited by a light source of a wavelength different from that used to excite PE or one of its derivatives, and that has an emission spectrum offset from that of PE or one of its derivatives.

This particular embodiment requires at least two light sources, one of a wavelength between 330 and 425 nm, preferably at 405 nm, making it possible to simultaneously excite PE and/or one of its tandems and the second light source of a longer wavelength such as for instance a laser at 530 nm or 560 nm or even a laser/diode at 635/640 nm, making it possible to excite a second fluorophore different from PE and/or one of its derivatives.

In all case, the skilled artisan shall avoid or minimise as much as possible that the light signal emitted by a fluorophore overflows in the detection channel that should record the signal emitted by another fluorochrome. The limitation of spectral overlap of 2, 3, 4, 5 or umpteen fluorochromes allows for the reliable detection of several molecules of interest. This method is currently applied in particular with a laser at 488 nm that allows for the routine measurement of 5 different molecules stained by different antibodies and fluorochromes (e.g. FITC, PE, PE-Texas-Red or ECD, PE-Cy5, PE-Cy7).

Step iv) of determining the presence or absence of the molecule of interest in the sample is based on comparing the results obtained in step iii) in this sample with those obtained for a reference sample known not to contain the molecule of interest.

Once the presence of the molecule of interest has been determined, step iv) can also be used to calculate the number of copies of the molecule of interest in the sample. This quantification step can be executed in comparison with a standard range containing increasing known quantities of the targeted molecule of interest.

The method for detecting at least one molecule of interest in a sample according to the invention can be implemented on a flow cytometer.

The invention detection method may be used for the separation, analysis or counting of cellular or particulate subpopulations in a biological sample.

The invention detection method may be used for the separation, analysis or counting of cellular or particulate subpopulations of haematopoietic origin.

A second object of the invention concerns the use of a fluorochrome consisting of phycoerythrin or one of its derivatives for the detection of at least one molecule of interest in a sample, characterised in that the excitation wavelength of the fluorochrome is between 330 nm and 425 nm, preferably between 350 and 420 nm, or even more preferably between 360 and 410 nm.

According to a particular embodiment, the excitation wavelength of the fluorochrome is between 400 and 410 nm, and preferably at 405 nm.

The invention is illustrated by the examples hereinafter, without the content of their teaching restricting the scope of the invention or constituting a limitation of sorts.

### EXAMPLES

### 1 - Genesis of the Invention

This invention was sparked by casual observation. More specifically, and during a typical experience relative to the setting of fluorescence compensation performed by an expert specialising in this type of flow cytometer with 3 lasers (BC Gallios™), a low parasitic leakage in one of the detectors associated with the violet laser (FL10 channel) was observed by the inventor. In this particular case, a mouse Ig capture bead, of the type described in example 2 and stained only with a PE conjugate provided a significant signal level in FL10 (fluorescence between 530 and 570 nm, 550 BP40 filter), where theory expected a total lack of fluorescence in the absence of a conjugate stained with Krome-Orange™, fluorophore compatible with this range of excitation and emission wavelengths. The inventor, who did not feel this observation to be insignificant, aimed to glean the reason behind this leakage.

To this end, it was necessary to i) dispose of a similar system, namely a bead only stained with PE and ii) alter the standard structure of the optical bench of this device by shifting some filters. The different filters of the optical bench were manually shifted to another position so as to allow each filter to pass in front of the FL10 detector associated with excitation at 405 nm. These modifications made it possible to ensure that the maximum signal was obtained with the optimised filter for PE (575 BP 30), normally installed with the blue laser at 488 nm and on the FL2 detector. Accordingly, the results of this test suggested that it could be a fluorescent signal from the PE, the sole fluorochrome present on this type of bead but in the unexpected context of an excitation at 405 nm.

For the ensuing verifications, a number of unconventional modifications of the device were necessary. These modifications aimed to enable the reading of PE fluorescence simultaneously on channels FL2 (normal position associated with excitation at 488 nm, as instructed in the operation manuals) and FL10 (abnormal position, associated with excitation at 405 nm), which could only be executed with the availability in the same place of two similar devices, whereby the PE filter of the second Gallios™ cytometer was promptly borrowed and placed in the FL10 position of the first Gallios™ cytometer. These anachronic manipulations made it possible, as shown by the examples hereinafter, to illustrate the possibility of measuring varying levels of PE fluorescence under excitation at 405 nm with striking efficacy and a resolution close to that obtained with the conventional excitation at 488 nm.

### 2 - A Study of the Cross-contamination of Inter-Laser Fluorescence

Some analytical devices, such as flow cytometers or cell sorters, can have up to 6 extra lasers in addition to the violet laser at 405 nm. Today, these instruments are currently equipped with 3 lasers, namely i) a blue laser at 488 nm, viewed as the laser providing temporal reference and analysing scattering together with multiple fluorescences ii) a red laser at 640 nm that illuminates the cells underneath/after the reference illumination point, iii) a violet laser at 405 nm that illuminates the cells above/before the reference illumination point of the blue laser. This illumination in multiple points enables a double separation, optical and temporal of the fluorescences from each illumination points. Indeed, the signal from each of the 3 lasers undergoes i) a shift in time by approximately a few tens of microseconds (typically between 30 and 40 µs) and ii) a shift in space of a distance equal to approximately a few hundreds micrometers (typically 100 µm), allowing for their differential focusing via the collection optics towards different optical fibers (one per laser). Thus, it is typically considered in multi-colour FCM, that the fluorescence from one of the three lasers are essentially devoid of optical contamination from one of the other two lasers.

### 2.1 - Principle

The operation of a multi-laser cytometer involves the excitation of the particles to be analysed for each of the lasers with a shift in space and time. Each cell/particle sequentially passes through some light beams. For the GALLIOS™/NAVIOS cytometer (Beckman-Coulter, Villepinte, F) equipped with 3 lasers (violet - blue - red, namely at 405-488-640 nm), the order is as follows: violet - blue - red, with a distance between the impact points equal to ~100 µm and a time equal to ~30 µs. The lens collecting the light signals at 90° (« pick-up lens ») differentially collects the light emitted at the 3 impact points and focuses on 3 independent optical fiber inlets that then guide the light towards an optical system able to independently separate the wavelengths from each optical fiber.

Each laser routinely operates at a fixed nominal power (violet at 40 mW- blue at 22 mW - red at 25 mW). By intervening on the GALLIOS™ Service module, it is possible to intentionally modify the power of each laser and therefore examine the impact of the excitation power on the signals measured by the cytometer.

This principle was drawn upon to prove that PE is indeed excitable by a violet laser (405 nm) and to rule out any risk of artefact that could derive from a cross-contamination of inter-laser fluorescences, and specifically a fluorescence leakage induced by the blue laser towards the detectors coupled to the violet laser.

### 2.2 - Calibrators

10 µm- diameter polystyrene beads covered in varying quantities of PE serve as calibrators: beads C1, C2, C3 and C4 respectively have 900, 7,500, 115,000 and 1,250,000 R-PE molecules/bead. Two lots of beads were synthesised and suspended in a solution composed of PBS + BSA 0.1% + sodium azide 0.1%.

### 2.3 - Protocol

The signal of the blue laser gives t₀ for one particle and the signals associated with this same particle by the other lasers are considered respectively at t₀-30 µs (violet laser) and t₀+30 µs (red laser).

Aside from the benchmarking analyses conducted in standard conditions in terms of laser power values (violet 40 mW- blue 22 mW - red 25 mW), the diverse power values of the following were tested:
Blue laser : 22- 20-10-5-3-1 mW
Violet laser : 40 - 30 - 20 - 5 - 1 mW

In some cases, it is also possible to turn off the violet laser (via a software-controlled iris) during analysis. At this point, the blue laser must always remain functional in that it is used as t₀. Its power can be decreased but not entirely blocked, unlike the other 2 lasers.

The arrangement of the filters of the optical bench was modified in order to optimise the detection of PE fluorescence signals transmitted via the fibers coupled to the respective focal points of the blue and violet lasers. In this particular installation, two identical copies of a 575 BP 30 bandpass filter, optimal for PE, are used in parallel, one in front of the FL2 detector and the other in front of FL10. The FL2 channel therefore measures the PE signals from the blue laser (488 nm) passing through the 575 BP 30 filter, behind a 595 DC SP dichroic mirror. FL10 corresponds to the PE signals from the violet laser (405 nm) passing through an identical 575 BP 30 filter, after deflection via a 480 DC SP dichroic mirror.

### 2.4 - Results

The nominal powers of the blue and violet lasers are respectively equal to 22 mW and 40 mW (FIG. 2, 4-decades scale and FIG. 5, 5-decades scale). The bead singlets are initially preselected by the «beads» region in a double scatter analysis and their FL2 and FL10 fluorescence are analysed accordingly.

### Modulation of the Blue Laser (488 Nm)

The nominal power of the blue laser is decreased to 10 mW (FIG. 3) and then 5 mW (FIG. 4), whereas the nominal power of the violet laser is maintained at 40 mW.

As illustrated in FIG. 2, it is possible to observe:
An extremely clear separation of the 4 peaks in FL2, median ratios equal to or higher than 10.
A separation that is effective but slightly less marked in FL10 (with maximum median ratios = 8), and especially in the lowest PE density levels.

The drop in power of the blue laser from 22 mW to 10 mW (FIG. 3) and then to 5 mW (FIG. 4) decreases the intensity of all peaks in FL2 (blue laser) without any impact on fluorescence in FL10 (violet laser). This effect is confirmed for power values less than 5 mW, specifically 3 mW or even 1 mW (not shown).

Similar results were obtained on a second lot of calibration beads (not shown). The drop in maximum nominal power of the blue laser (decreased to 1 mW) causes a change of scale on the histograms for an optimal analysis (5 decades instead of 4).

### Modulation of the Violet Laser (405 Nm)

The nominal power of the violet laser is decreased to 10 mW (FIG. 6) and then 5 mW (FIG. 7), whereas the nominal power of the blue laser is maintained at 22 mW.

The reference (FIG. 5) remains identical to FIG. 2 but with the use of a 5-decades scale.

As illustrated in FIGS. 6 and 7, the drop in power of the violet laser from 40 mW (FIG. 5) to 10 mW (FIG. 6) and then to 5 mW (FIG. 7) decreases the intensity of all peaks in FL10 (PE from the violet laser) without any impact on fluorescence in FL2 (PE from the blue laser).

Furthermore, blocking the violet laser at 0 mW via an iris during analysis (FIG. 8) results in an image bringing together an assortment of PE fluorescence (FL2 and FL10) viewed in both conditions, before and after blocking. For each subfamily of PE beads, it is possible to observe the drastic fall in intensity of FL10 for the open versus closed violet laser, whereas that of FL2 remains unaltered.

In conclusion, the study of the impact of the laser power on the distribution of PE fluorescence of a calibrant with 4 PE loading levels shows:
1) That the modulations of the blue laser only impact the FL2 resolution (PE from the blue laser)
2) That the modulations of the violet laser only impact the FL10 resolution (PE from the violet laser)

This eliminates any risk of artefact that could derive from a cross-contamination of inter-laser fluorescences, and specifically a PE fluorescence leakage induced by the blue laser towards the detectors coupled to the violet laser.

Lastly, the linearity of the obtained profiles makes it possible to validate the fact that PE fluorescence is the result of an excitation at 405 nm and therefore confirms the possible use of PE as a fluorochrome with a laser at 405 nm.

### 3 - Research Study Relative to the Excitation of Tandems on PE With a Laser at 405 nm (and/or 488 nm)

### 3.1 - Principle

Mouse Ig (immunoglobulin) capture beads are stained with different PE conjugates and/or PE tandems. The 3 staining levels thus achieved are analysed by using the same passband filter on FL2, FL4 or FL5 respectively (excitation at 488 nm of reference for PE, PE-Cy5 and PE-Cy7 respectively) and on FL10 (excitation at 405 nm tested, filtered with the same filter as the one used for PE, PE-Cy5 and PE-Cy7 respectively).

### 3.2 - Capture Beads

Mouse Ig capture beads (H+L) with a diameter of 7 µm (SPHERO-COMPtrol Particles, ref. CMIgP-70-3K) marketed by SPHEROTECH, Inc. are used in compliance with the supplier instructions. The three bead populations correspond to a negative control, one bead poorly covered and one bead heavily covered with the goat anti-mouse antibody and accordingly, with the conjugated mouse Ig.

### 3.3 - Protocol

The capture beads are incubated with 20 µL of conjugate (mouse antibody coupled to PE or one of its derivatives, PE-Cy5, PE-Cy5.5 or PE-Cy7) at ambient temperature, in the dark for 1 h. Then, 500 µL of PBS-BSA buffer is added.

The same passband filter as that of the optimal FLi of the blue laser (i.e. FL2, 4 or 5) is taken from a second Gallios™cytometer similar to that of the test and placed on the FL10 channel.

### 3.4 - Results

As detailed in FIG. 9, it is possible to observe a clear separation of all 3 peaks not only in FL4, but also in FL10 for a mouse antibody conjugated to the PE-Cy5.5 tandem. Similar results were obtained for mouse antibodies conjugated with PE, PE-Cy5 and PE-Cy7.

The table below elucidates the full results obtained for the 4 tested antibodies.

| FL2, 4 or 5 | PE | PE-CY5 | PE-CY5.5 | PE-CY7 |
|---|---|---|---|---|
| H | 0.3 | 1.6 | 0.9 | 4.7 |
| I | 1.6 | 7.1 | 8.5 | 16.9 |
| J | 46.3 | 154.5 | 194.1 | 330.1 |
| R1=J/I | 29 | 22 | 23 | 20 |
| R2=I/H | 5 | 4 | 9 | 4 |
| R3=J/H | 154 | 97 | 216 | 70 |

| | | | | |
|---|---|---|---|---|
| FL10 | PE | PE-CY5 | PE-CY5.5 | PE-CY7 |
| AH | 1.4 | 2.9 | 1 | 1.1 |
| AI | 2.5 | 9.3 | 4.3 | 3 |
| AJ | 41.2 | 171.2 | 85 | 54.3 |
| R1= AJ/AI | 16 | 18 | 20 | 18 |
| R2= AI/AH | 2 | 3 | 4 | 3 |
| R3= AJ/AH | 29 | 59 | 85 | 49 |

The calculation of the R1, R2 and R3 ratios accounts for, respectively, the ability to discriminate:
R1: a strongly stained vs a lightly stained sample,
R1: a lightly stained vs an unstained sample,
R1: a strongly stained vs an unstained sample,

The values of the ratios clearly show that the excitation of PE, PE-Cy5, PE-Cy5.5 and PE-Cy7 by a violet laser allows for an excellent discrimination between a strongly stained vs a lightly stained sample, and particularly between a strongly stained vs an unstained sample. Although to a lesser extent as compared to what is observed upon excitation by the blue laser, it is also possible to discriminate a lightly stained vs an unstained sample.

### 4 - Identification of Cell Subpopulations

### 4.1 - Sample

The model used consists in EDTA whole blood (WB) containing an abnormal cell type represented here by the LP1 multiple myeloma cell line. This model outlines a case of detection of leukaemic haematopoietic cells that in this case are similar to blasts.

LP1 cells have been added to the whole blood sample at a 10% concentration as compared to the total white blood cells.

### 4.2 - Cell Marking

The characterisation of the different cellular populations present in the sample was carried out thanks to an anti-CD45 monoclonal antibody conjugated to PE (BioCytex). The density of CD45 is approximately 200,000 molecules/lymphocyte versus 120,000 molecules/monocyte, respectively, 217± 64 x 10³ vs 103 ± 44 x 10³, according to BIKOUE et al, (Cytometry, 26:137-147 (1996)).

The technique consists in incubating the whole blood cells in the presence of anti-CD45-PE. The red blood cells are then lysed, the remaining cells (white blood cells) are washed to remove the unbound fluorescent conjugate, and then analysed by flow cytometry.

### 4.3 - Flow Cytometry Analysis

In our experience and in literature, the blasts routinely searched for in blood counts will, most often, be better discriminated by this CD45 x SSC system than the LP1 cells of this example, especially if other parameters (volume, FSC) contribute to it.

As the LP1 cells are not entirely isolated from the normal granulocytes (PMNs) in a CD45 x SSC graph, we drew upon a complementary parameter i.e. a blue-light autofluorescence of the LP1 cells in FL9 (Ex 405 nm / Em 460/20 nm) higher than those of normal blood cells that facilitates their detection and the colourisation of the illustrated subpopulations.

Typically, the «side scatter» (SS) parameter allows for the identification of the 3 major leukocyte subpopulations -i.e. lymphocytes, monocytes and granulocytes (polymorphonuclear neutrophils also referred to as PMNs). The LP1 cells are added on to the usual scheme, positioned at the same SS level as the PMNs, and above the PMNs as a «forward scatter» (FS) parameter. The FS x SS graph also helps to provide an analysis window for the residual red blood cells (RBC), remaining after lysis.

The stained cells were analysed on a Gallios™ flow cytometer (Beckman Coulter). The analysis was conducted on approximately 30 µl of stained sample, acquiring at least 7,000 events at a speed of 60 µL/min. for 30 seconds. The data were analysed using the KALUZA® computer software (Beckman Coulter). The voltages of the FL2 and FL10 PMTs were respectively equal to 350 V and 420 V and the light signals filtered by the same type of 575 nm/30 filter (FL10 standard filter replaced by a 575 nm/30 filter) so as to collect on FL2 and FL10 the PE fluorescence excited respectively by the blue laser at 488 nm and the violet laser at 405 nm.

First and foremost, the analysis strategy includes the conditioning of all cells in a size/structure analysis window. The PE fluorescence of the different cellular subpopulations may be visualised on a bi-parametric graph representing the fluorescence intensity on the y-axis as a function of a parameter correlated to cell granularity (SS) and that allows for a first classification of leukocytes in order of SS intensity, i.e. from left to right, lymphocytes, monocytes and granulocytes/PMNs (and LP1 in case of overload in LP1) and the number of events on the x-axis. The software provides a mean fluorescence intensity (MFI) for each tube.

The cells of the LP1 cell line are also isolated by the association of two parameters, i.e. their size, viewed on the FS parameter (more important than for the other leukocytes) and their autofluorescence detectable in FL9 (460 nm +/- 20 nm).

### 4.4 - Results

### Normal Blood

As illustrated in FIGS. 10 (A) to (B), the comparison of the CD45-PE vs SS graphs indicates somewhat similar cell distributions, regardless of whether PE is excited by the blue laser (488 nm/FL2) or by the violet laser (405 nm/FL10). The 2 conditions allow a good CD45 vs SS discrimination of the major cellular subpopulations of the whole blood.

The representation in combined mono-parametric histograms of the 4 subpopulations (Lymphocytes, monocytes, granulocytes and residual red blood cells, FIGS. 10 (E) and (F)) shows that the order of CD45 staining intensities is complied with.

Following PE excitation with either one of the two lasers and collection of the light emitted on a filter optimal for PE (Ex 488 nm / Em 575/30 nm in standard condition and Ex 405 nm / Em 575/30 nm in tested condition), the ratio between the MFIs of cellular subpopulations is calculated and detailed in the table below.

| Ratios | 488 nm laser | 405 nm laser |
|---|---|---|
| Lymphocytes/Monocytes | 41/19.7 = 2.1 | 78.3/39.1 = 2.0 |
| Lymphocytes/PMNs | 41/5.9 = 7.0 | 78.3/14 = 5.6 |
| Lymphocytes/RBCs | 41/0.6 = 68.3 | 78.3/2.1 = 37.3 |
| Monocytes/PMNs | 19.7/5.9 = 3.3 | 39.1/14 = 2.8 |
| Monocytes/RBCs | 19.7/0.6 = 32.7 | 39.1/2.1 = 18.6 |
| PMNs/RBCs | 5.9/0.6 = 9.8 | 14/2.1 = 6.7 |

It may be observed that, even if they are slightly lower in the 405 nm system, these ratios are maintained and the MFIs tiering between the subpopulations is respected.

This similar MFI tiering in both the 488 nm and 405 nm systems results in ratios between subpopulations that are quite close. The greater rangeability of the standard system with PE excitation at 488 nm (as compared to the PE excitation system at 405 nm discovered herein) may only be truly observed when an unstained subpopulation (RBCs) is compared to stained subpopulations, e.g. 68 vs 37 Lymphocytes/RBCs ratios.

### Blood Coated With Leukaemic Blasts

When the CD45 vs SS analysis is pre-conditioned to reveal the leukaemic blasts (LP1), a cloud-shaped modification of the PMNs may be observed, that is similar in both systems i.e. excitation of CD45-PE at 488 nm ( FIG. 11 (C) ) or at 405 nm (FIG. 11 (D)), thus suggesting that both systems offer the possibility to detect pathological cells present in the blood characterised by an abnormal level of CD45 expression. Moreover, modifications on the CD45 x SS graph of the blood suggest that the LP1 cells are positioned in a cloud similar to that of the granulocytes (and in partial superposition).

The superposition of mono-parametric histograms relative to CD45-PE staining on the set of subpopulations of this blood sample (FIGS. 11 (E) and (F)) outlines the similarities of staining distribution in the 2 systems at 488 nm and at 405 nm. It may be observed that the LP1 histogram is superposed on the PMN histogram.

Following PE excitation using either one of the two lasers and collection of the light emitted on a filter optimal for PE (Ex 488 nm / Em 575/30 nm standard condition and Ex 405 nm / Em 575/30 nm tested condition), the ratio between the MFIs of normal and leukaemic cellular subpopulations is calculated and detailed in the table below.

| Ratios | 488 nm laser | 405 nm laser |
|---|---|---|
| Lymphocytes/Monocytes | 41/19.7 = 2.1 | 78.3/39.1 = 2.0 |
| Lymphocytes/PMNs | 41/5.9 = 7.0 | 78.3/14 = 5.6 |
| Lymphocytes/RBCs | 41/0.6 = 68.3 | 78.3/2.1 = 37.3 |
| Monocytes/PMNs | 19.7/5.9 = 3.3 | 39.1/14 = 2.8 |
| Ratios | 488 nm laser | 405 nm laser |
| Monocytes/RBCs | 19.7/0.6 = 32.7 | 39.1/2.1 = 18.6 |
| PMNs/RBCs | 5.9/0.6 = 9.8 | 14/2.1 = 6.7 |
| Lymphocytes/Blasts | 41/5.2 = 7.9 | 78.3/13.4 = 5.8 |
| Monocytes/Blasts | 19.7/5.2 = 3.8 | 39.1/13.4 = 2.9 |
| PMNs/Blasts | 5.9/5.2 = 1.13 | 14/13.4 = 1.04 |
| Blasts/RBCs | 5.2/0.6 = 8.7 | 13.4/2.1 = 6.4 |

Again, the MFI ratios between blasts and normal blood cells are fairly similar in the 2 systems at 488 nm and at 405 nm, thus showing that the diagnostic discrimination of blood cell subpopulations is feasible even by exciting PE at 405 nm

All in all, the observed discrimination, following independent PE excitation by a blue laser at 488 nm (reference) and by a violet laser at 405 nm, between cell subpopulations stained at different levels by this fluorochrome show that PE can be used as a fluorochrome with a violet laser, thus allowing for useful discrimination between cellular subpopulations of interest, such as, for instance, subpopulations of normal blood leukocytes and leukaemic cells after staining with a CD45 antibody.

### 5 - Identification of Leukaemic Blasts

### 5.1 - Sample

The model used consists in EDTA whole blood (WB) containing two abnormal cell types represented here by the LAM HL60 and NB4 lines. These models outline a case of detection of leukaemic haematopoietic cells that in this case are similar to blasts.

HL60 and NB4 cells were mixed at a concentration equal to 5 million/ml in PBS-BA. 10 µl of this mixture were added to a 50 µl sample of whole blood, that is to say 50,000 cells of the HL60 and NB4 mixture for 50 µl of whole blood.

### 5.2 - Cell Staining

The characterisation of the different cellular subpopulations present in the sample was performed thanks to an anti-CD45 monoclonal antibody conjugated to PE (BioCytex) used alone or in multicolour staining in the presence of other fluorescent reagents, of which an anti-CD33 monoclonal antibody conjugated to PE-Cy7.

The technique consists in incubating the whole blood spiked with HL60 and NB4 cells in the presence of the anti-CD45-PE alone or in combination with the anti-CD33 conjugated to PE-Cy7. The red blood cells are then partially lysed, the remaining cells are washed to remove the unbound fluorescent conjugate/s, then analysed by flow cytometry.

The efficacy of the lysis of the red blood cells was intentionally limited so as to retain in the analysis a considerable presence of residual red blood cells, useful for the experiment. Indeed, the residual red blood cells are, by definition, negative for all tested markers.

### 5.3 - Flow Cytometry Analysis

In our experience, the cellular subpopulations (normal cells and leukaemic blasts) are more often than not discriminated by the CD45 x SSC system (not shown here) or by the CD33 x SSC system (FIGS. 12A and B) and the CD33xCD45 system (FIGS. 12C and D).

The stained cells were analysed on a Gallios™flow cytometer (Beckman Coulter). The analysis was conducted on approximately 30 µl of stained sample, acquiring at least 7,000 events at a speed of 60 µL/min. for 30 seconds. The data were analysed using the KALUZA® computer software (Beckman Coulter). The voltages of the FL5 and FL10 PMTs were respectively equal to 600 V and 900 V and the light signals filtered by the same type of 755 nm long pass filter (the FL10 standard filter was substituted by a 2^{nd}755 nm long pass filter identical to the one placed in FL5) so as to collect on FL5 and FL10 the PE-Cy7 fluorescence excited respectively by the blue laser at 488 nm and the violet laser at 405 nm.

First and foremost, the analysis strategy includes the conditioning of all cells in a size/structure analysis window. The PE or PE-Cy7 fluorescence of the different cell subpopulations may be visualised on a bi-parametric graph representing the fluorescence intensity on the y-axis as a function of a parameter correlated to cell granularity (SS) on the x-axis, allowing for a first classification of leukocytes in order of SS intensity, i.e. from left to right, lymphocytes, monocytes and granulocytes/PMNs (and blasts in case of overload in HL60 + NB4). For each cell type defined on the graph by a region of interest, the software provides a percentage of all cells present in the graph and a mean fluorescence intensity (MFI, noted as «Y-Med»).

During multicolour staining (including CD45-PE and CD33-PE-Cy7), a bi-parametric graph representing the corresponding fluorescence intensities, respectively on the x-axis and on the y-axis, also allows to view the different cellular subpopulations.

### 5.4 - Results

As illustrated in FIGS. 12 (A) and (B), the comparison of the CD33-PE-Cy7 (CD33-PC7) vs SS graphs displays similar cellular distributions, regardless of whether PE-Cy7 (PC7) is excited by the blue laser (488 nm/FL5) or by the violet laser (405 nm/FL10). The two excitation conditions allow for a good CD33 vs SS discrimination of the major cellular subpopulations (lymphocytes, monocytes, PMNs and residual red blood cells) of the whole blood as well as of the leukaemic blasts (HL60 and NB4). Moreover, the blasts are also discriminated by the level of CD33 expression: HL60 cells have a level of CD33 expression higher than that observed for NB4 cells.

Following PE-Cy7 excitation using either one of the two lasers and collection of the light emitted on a filter optimal for PE-Cy7 (Ex 488 nm / Em 755 nm long pass in standard condition and Ex 405 nm / Em 755 nm long pass in tested condition), the ratio between the MFIs of normal and leukaemic cellular subpopulations is calculated and detailed in the table below.

| Ratios | 488 nm laser | 405 nm laser |
|---|---|---|
| Neutrophils/Lymphocytes | 147/0.86 = 171 | 155.7/2.1 = 74 |
| Neutrophils/NB4 | 147/9.1 = 16 | 155.7/17.1 = 9.1 |
| Neutrophils/HL60 | 147/74.5 = 2 | 155.7/74.2 = 2.1 |
| Neutrophils/Monocytes | 147/15 = 9.8 | 155.7/14.5 = 10.7 |
| NB4/Lymphocytes | 9.1/0.86 = 10.6 | 17.1/2.1 = 8.1 |

Again, the MFI ratios between blasts and normal blood cells are fairly similar in the 2 systems at 488 nm and at 405 nm, thus showing that the diagnostic discrimination of normal and leukaemic blood cell subpopulations is feasible even by exciting PE-Cy7 at 405 nm.

All in all, the discriminations observed, following independent PE-Cy7 excitation by a blue laser at 488 nm (reference) and by a violet laser at 405 nm, between cell subpopulations stained at different levels by this fluorochrome show that PE-Cy7 can be used as a fluorochrome with a violet laser, allowing for useful discriminations between cellular subpopulations of interest, such as, for instance, subpopulations of normal blood leukocytes and leukaemic cells after staining with a CD33 antibody.

Furthermore, as illustrated in FIGS. 12 (C) and (D), the comparison of the CD33-PE-Cy7 (FL5) vs CD45-PE (FL2) graphs or CD33-PE-Cy7 (FL10) vs CD45-PE (FL2) graphs shows similar cellular distributions, whether the fluorescence measurement is executed in FL5 (exc.488 nm) or in FL10 (exc. 405 nm). This bi-parametric analysis also allows for an enhanced discrimination of two sub-populations of neutrophils (Neu and PMN2) based on their level of CD33 expression. The discrimination of the residual red blood cells and lymphocytes based on the level of CD45 expression is also enhanced.

These findings show that PE derivatives, illustrated herein by PE-Cy7, are usable at an excitation wavelength of 405 nm.

1. A method for detecting at least one molecule of interest in a sample, comprising the steps of:
i) Contacting the sample with a ligand that specifically binds the molecule of interest, said ligand being conjugated to a fluorophore consisting of phycoerythrin or a derivative thereof,
ii) Exciting the mixture of step i) by at least one light source with a wavelength range between 400 and 410 nm,
iii) Detecting light emitted by the mixture excited in step ii) at a wavelength longer than or equal to 550 nm, and
iv) Determining the presence or absence of the molecule of interest in the sample based on the results of step iii) .

2. The method according to claim 1, wherein said at least one molecule of interest is a protein, a lipid, a carbohydrate, a lipid or glycolipid structure or a carbohydrate unit.

3. The method according to claim 1, wherein the sample comprises or is likely to comprise cells.

4. The method according to claim 1, wherein the specific ligand of said at least one molecule of interest is an antibody.

5. The method according to claim 1, wherein the fluorophore is R-phycoerythrin (R-PE) .

6. The method according to claim 1, wherein the at least one light source used to excite the mixture of step i) is a violet laser or a violet laser diode emitting at a wavelength of 405 nm.

7. The method according to claim 1, further comprising the simultaneous identification of at least one second molecule of interest in the sample.

8. The method according to claim 1, that is implemented on a flow cytometer.

9. The method according to claim 1, further comprising separating, analysing or counting of cellular or particulate subpopulations in a biological sample.

10. The method according to claim 9, wherein the cellular or particulate subpopulations are of haematopoietic origin.

11. The method according to claim 2, wherein the sample comprises or is likely to comprise cells.

12. The method according to claim 2, wherein the specific ligand of said at least one molecule of interest is an antibody.

13. The method according to claim 3, wherein the specific ligand of said at least one molecule of interest is an antibody.

14. The method according to claim 2, wherein the fluorophore is R-phycoerythrin (R-PE).

15. The method according to claim 3, wherein the fluorophore is R-phycoerythrin (R-PE).

16. The method according to claim 4, wherein the fluorophore is R-phycoerythrin (R-PE).

17. The method according to claim 2, further comprising the simultaneous identification of at least one second molecule of interest in the sample.

18. The method according to claim 3, further comprising the simultaneous identification of at least one second molecule of interest in the sample.

19. The method according to claim 4, further comprising the simultaneous identification of at least one second molecule of interest in the sample.

20. The method according to claim 5, further comprising the simultaneous identification of at least one second molecule of interest in the sample.
